(19) United States
(12) Patent Application Publication
(10) Pub. No.: US 2009/0186768 A1
(43) Pub. Date: Jul. 23, 2009

(54) Sunscreen formulations for use in the production of organic crops

(76) Inventors: Ray J. Hoobler, Pleasanton, CA (US); David T. Brantner, Livermore, CA (US)

Correspondence Address: CARR & FERRELL LLP, 2200 GENG ROAD, PALO ALTO, CA, 94303, US

(21) Appl. No.: 12/291,875

(22) Filed: Nov. 14, 2008

Related U.S. Application Data
(60) Provisional application No. 61/003,347, filed on Nov. 16, 2007.
(60) Provisional application No. 61/003,782, filed on Nov. 19, 2007.

(51) Int. Cl. A01N 25/02; C12N 5/04; C09K 3/00

(52) U.S. Cl. 504/362; 435/410; 252/383

(57) ABSTRACT

Provided herein are exemplary agricultural sunscreen formulations for use in the production of organic crops. One exemplary agricultural sunscreen formulation comprises on a weight to weight basis about 40% to 80% calcium carbonate, about 1% to 5% sodium bicarbonate, and about 15% to 59% water. Also provided herein are exemplary methods for making an exemplary agricultural sunscreen formulation for use in the production of organic crops. One exemplary method comprises combining a first amount of glycerin and an amount of water to form a first mixture, combining an amount of particles with the first mixture, combining an amount of a thickening agent and a second amount of glycerin to form a second mixture, and combining the first and second mixtures.

### CROSS-REFERENCE TO RELATED APPLICATION

This application claims the benefit and priority of U.S. Provisional Patent Application Ser. No. 61/003,347 filed on Nov. 16, 2007, titled “Organic Sunscreen Formulation for Plant Tissue,” which is hereby incorporated by reference.

This application is related to U.S. Provisional Patent Application Ser. No. 61/003,782 filed on Nov. 19, 2007 titled “Using Particle Films to Reduce Stress in Plant Tissue,” and U.S. patent application Ser. No. ______ filed on Nov. ______, 2008 titled “Systems and Methods for Applying Particle Films to Control Stress on Plant Tissues,” both of which are incorporated herein by reference.

### BACKGROUND OF THE INVENTION

1. Field of the Invention

This invention relates to managing stress on plant tissues, and more specifically to sunscreen formulations for use in the production of organic crops.

2. Description of Related Art

Organic crops are generally grown with organic materials. Organic crops may also be required to be grown using energy-saving technologies and packaged using recyclable or biodegradable materials when possible. Currently available sunscreen formulations do not meet the criteria for use in the production of organic crops. Some of these sunscreen formulations employ chemicals that are not approved for use in the production of organic crops. Other sunscreen formulations contain ingredients having undesirable contaminants, such as lead, cadmium, fluoride, arsenic, aluminum and/or silicon. Additionally, some sunscreen formulations use non-organic chemicals to change the physical properties of the sunscreen formulation. For example, some sunscreen formulations may employ non-organic chemicals to make the sunscreen formulation more hydrophobic.

Some of the challenges in creating a sunscreen formulation suitable for use in the production of organic crops include innovating a formulation that allows it to be manufactured, stored and shipped to customers with little or no degradation, maintained as a stable and non-perishable emulsion, applied with currently available spray equipment, and capable of achieving effective coverage of plant tissues. Accordingly, there is a need for sunscreen formulations for use in the production of organic crops.

### SUMMARY OF THE INVENTION

Provided herein are exemplary agricultural sunscreen formulations for use in the production of organic crops. One exemplary agricultural sunscreen formulation comprises on a weight to weight basis about 40% to 80% calcium carbonate, about 1% to 5% sodium bicarbonate, and about 15% to 59% water. Some exemplary agricultural sunscreen formulations may have a viscosity from about 1,000 to about 45,000 centipoise. Another exemplary agricultural sunscreen formulation may comprise on a weight to weight basis about 40% to 80% calcium carbonate, about 0% to 8% glycerin, about 0.1% to 0.5% potassium bicarbonate, and about 11.5% to 59.9% water.

Also provided herein are exemplary methods for making an exemplary agricultural sunscreen formulation for use in the production of organic crops. One exemplary method comprises combining a first amount of glycerin and an amount of water to form a first mixture, combining an amount of particles with the first mixture, combining an amount of a thickening agent and a second amount of glycerin to form a second mixture, and combining the first and second mixtures. According to various embodiments, the first amount of glycerin is from about zero to about three weight percent, and the particles are a micronized powder having a particle size distribution with a mean from about 0.01 to about 100 microns. Further methods may include sterilizing the particles before combining them with the first mixture. Another exemplary method for making an agricultural sunscreen formulation may comprise combining about 40% to 80% calcium carbonate with about 1% to 5% sodium bicarbonate and about 15% to 59% water to form an aqueous suspension.

### BRIEF DESCRIPTION OF THE DRAWINGS

FIG. 1 is a flow chart of a first exemplary method for making an exemplary agricultural sunscreen formulation for use in the production of organic crops.

FIG. 2 is a flow chart of a second exemplary method for making a second exemplary agricultural sunscreen formulation for use in the production of organic crops.

FIG. 3 is a flow chart of a third exemplary method for making a second exemplary agricultural sunscreen formulation for use in the production of organic crops.

### DETAILED DESCRIPTION OF THE INVENTION

Provided herein are exemplary agricultural sunscreen formulations for the production of organic crops. Also provided herein are exemplary methods for making exemplary agricultural sunscreen formulations for the production of organic crops. Organic generally refers to guidelines or criteria for labels such as, “Organic,” “Organic Certified,” “OMRI Listed,” and so on, as applied to organic crops. Such guidelines or criteria may be determined by organizations such as the Organic Materials Research Institute (OMRI), the National Organic Standards Board (NOSB), and/or other regulatory or advisory bodies.

For instance, the Organic Foods Production Act (OFPA) enacted under the 1990 Farm Bill served to establish uniform national standards for the production of crops and the handling of foods labeled as “Organic.” The OFPA established the NOSB which advises the Secretary of Agriculture in setting the standards upon which a National Organic Program (NOP) was established to set national standards for the production, handling, and processing of organically grown agricultural products. In addition, the NOP oversees mandatory certification of organic production. Organic food producers who meet standards set by the NOP may label their products as “USDA Certified Organic.” Outside of the United States, for a food product to be certified as organic, the food product must be grown and manufactured in a manner that adheres to standards set by the country in which the crops are sold.

An exemplary agricultural sunscreen formulation as provided herein for the production of organic crops may comprise an aqueous suspension having one or more of the following ingredients:

(i) a wetting agent to reduce interfacial tensions, allow efficient mixing of the ingredients of the sunscreen formulation, and facilitate uniform coverage of the surfaces of plant tissues by the sunscreen formulation;

(ii) a dispersion agent to preserve the state of the dispersion and prevent re-aggregation of the aqueous suspension;

(iii) an emulsifier to stabilize the aqueous suspension;

(iv) a preservative and/or a biocide to reduce microorganism populations or prevent microorganisms from growing; and

(v) an effective concentration of particles for forming a film that reduces transmission of ultraviolet (UV), visible (VIS) and/or near infrared (NIR) radiation.

In some exemplary embodiments, the particles are derived from mined materials of low solubility. For example, small calcium carbonate particles may be used as the active ingredient in the aqueous suspensions described herein. Calcium carbonate is generally a fine, white, microcrystalline powder which is stable in air, colorless and tasteless. Calcium carbonate is also very insoluble in water and alcohol. Micronized calcium carbonate powders may be most suitable, having mean particle sizes ranging from about 0.01 to about 100 microns, and preferably ranging from about 0.1 to about 5 microns. Calcium carbonate is non-combustible and has little or no potential for detrimental chemical interactions with other materials used in organic farming systems. Calcium carbonate (and its breakdown products) has little or no toxicity in the open environment and there is little or no probability of environmental contamination associated with the manufacture, use, misuse and/or disposal of mined calcium carbonate.

According to various exemplary embodiments, a small particle size has an advantageous sedimentation rate in an aqueous suspension. The small particle size also provides effective scattering of solar radiation. If the particles have a size larger than the preferred size, further embodiments of the present invention provide for reducing the original particle size to a smaller size. For example, a process known as wet milling using a Dyno®-Mill wet mill manufactured by Glen Mills, Inc. may be used to reduce the size of particles. The particle-size distribution may be very wide, depending on, for example, the quality of the starting particles. Thus, the desirable particle size in an aqueous suspension may be specified in terms of a significant number of particles (for example, more than 25 percent) having sizes that fall within a desired range.

Based on the weight of the aqueous suspension, some exemplary aqueous suspensions may contain from about 40 to about 80 percent weight/weight of calcium carbonate, and preferably from about 50 to about 70 percent weight/weight of calcium carbonate.

Further embodiments may include an effective amount of a suitable thickening agent, such as lecithin, xanthan gum, guar gum, smectite clay, and/or other materials with the desired properties. Preferably, from about 0.1 to about 0.5 percent weight/weight of the thickening agent may be used. The particular thickening agent or combination of thickening agents employed, and the amount used, may provide a viscosity range to the aqueous suspension of about 1,000 centipoise (cps) to about 45,000 cps, and preferably from about 10,000 to about 35,000 cps.

In various embodiments, an appropriate wetting agent, such as glycerin, may be used to enhance dispersion of the thickening agent in the aqueous suspension, and to facilitate effective coverage of plant tissues. From about one to about twenty, and preferably from about two to about 7.5 weight/weight percent of glycerin may be used. Under the NOP, glycerin produced by hydrolysis of vegetable oils is allowed as an ingredient that may be used in crop production and/or on processed products labeled as “organic.”

According to a further embodiment, the thickening agent may be dispersed in about 50 to about 100 percent glycerin in a separate container. After thorough mixing, a premix is formed that may be added to the other ingredients. Before adding the premix to the other ingredients, water may be added to the premix to hydrate the thickening agent. The amount of water used may be, for example, from about 0 percent to about eight percent weight/weight.

In some embodiments, an effective amount of one or more preservatives or biocides, such as Bacillus subtilis, potassium bicarbonate, sodium bicarbonate, and/or ethyl alcohol, may be included in the aqueous suspension. An effective amount of a preservative or a biocide in an aqueous suspension may range from about 0.01 to about five percent weight/weight. According to a further embodiment, the potential for growth of a microorganism in the aqueous suspension may be reduced or prevented by disinfecting and/or sterilizing the container in which the aqueous suspension or aqueous suspension concentrate is packaged and/or shipped.

According to a further embodiment, an adjuvant such as yucca extract may be used in the aqueous suspension. For example, the amount of yucca extract used may vary based on the amount of the materials in the aqueous suspension concentrate, but typically ranges from about 0 to about 3 percent weight/weight. Such an adjuvant may be a common agricultural adjuvant known by those skilled in the art. Such an adjuvant should be compatible for use in the production of organic crops.

In various exemplary embodiments, the aqueous suspension is in the form of a concentrate for dilution before use. For example, the aqueous suspension concentrate may be added to an appropriate amount of water. The amount of water used may vary based on the amount of the materials in the aqueous suspension concentrate, but typically ranges from about 10 to about 60 percent weight/weight, and preferably from about 20 percent to about 40 percent weight/weight.

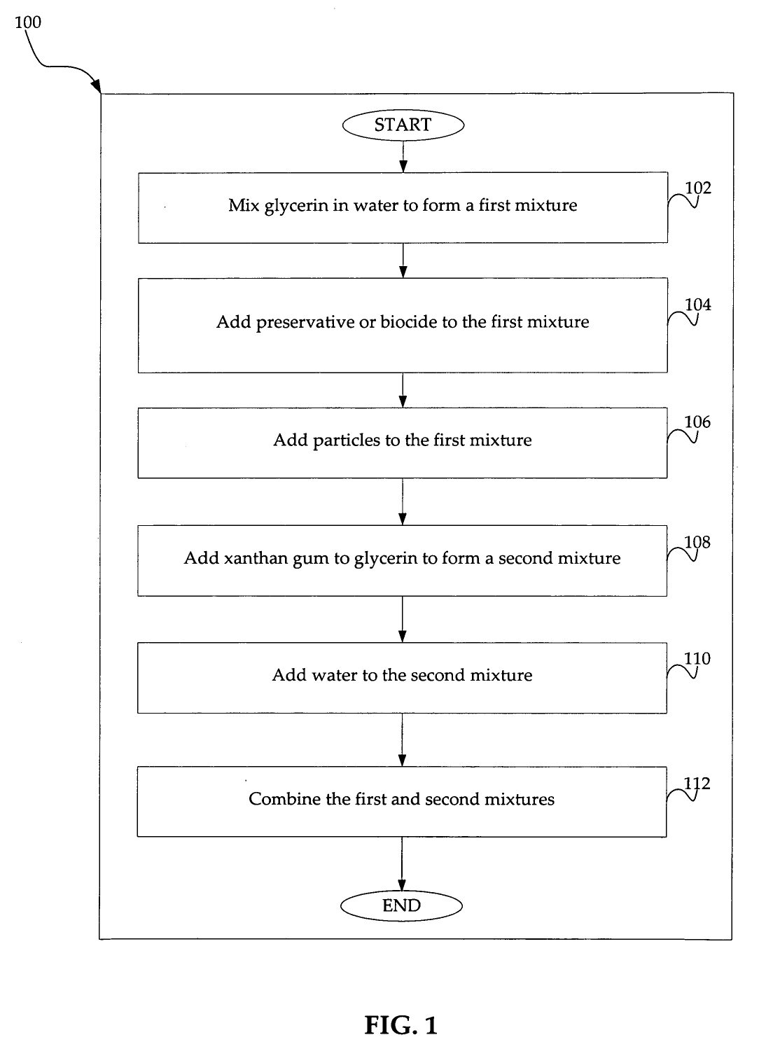

FIG. 1 is a flow chart of a first exemplary method 100 for making an exemplary agricultural sunscreen formulation for use in the production of organic crops.

At step 102, from about zero to about three weight percent of glycerin is mixed in water until the glycerin is well dispersed to form a first mixture.

At step 104, from about 0.01 to about five weight percent of a suitable preservative or biocide is added to the mixture of step 102.

At step 106, particles of low solubility, in the form of a micronized powder having a particle size distribution with a mean from about 0.01 to about 100 microns (and preferably from about 0.1 to about five microns), is added to the mixture of step 104. The particulate material should be completely wetted and dispersed in the first mixture. Optionally, the mean particle size may be reduced to less than one micron before or after performing step 106 (e.g., by wet milling).

At step 108, from about 0.1 to about 0.5 weight percent of xanthan gum is added to glycerin to form a second mixture or premix. Adequate mixing should be performed until the xanthan gum is well dispersed in the glycerin. Alternatively, from about zero to about five weight percent of xanthan gum may be added to the glycerin to form the second mixture or the premix.

At step 110, from about zero to about eight weight percent of water is added to the mixture of step 108.

At step 112, the mixture of step 110 is added to the mixture of step 106 to form a uniform aqueous suspension concentrate. Please note: standard methods of mixing, suspension, and/or dispersion may be used at each of the above steps.

The method exemplified in FIG. 1 may also include additional steps for sterilization, such as sterilizing the particles by exposing them to chemicals such as ethyl alcohol, hydrogen peroxide, ethanol and/or gaseous ozone prior to performing the method of step 106. Additionally, the particles may be heated to a temperature of at least 30 degrees Centigrade, and preferably to a temperature of at least 90 degrees Centigrade before performing the method of step 106. The various mixtures described herein may be exposed to ozone, ultraviolet light, pasteurization processes, and/or other sterilization techniques. Packaging materials may also be sterilized.

Table 1 shows an exemplary sunscreen formulation. All percentages are shown on a weight/weight (grams/grams) basis. Some or all of the ingredients may comply with the requirements for the production of organic crops.

TABLE 1

| | Percent (w/w) | |
|---|---|---|
| | Exemplary Range | Preferred Range |
| | (from about first value | (from about first value |
| Ingredients | to about second value) | to about second value) |
| Calcium Carbonate | 40.0-80.0 | 50.0-70.0 |
| Glycerin | 0-8.0 | 2.0-7.0 |
| Xanthan Gum | 0.1-0.5 | 0.1-0.5 |
| Potassium Bicarbonate | 0.01-5.0 | 0.5-1.0 |
| Water Q.S. | 100 | 100 |
| Viscosity | 1,000-45,000 cps | 10,000-35,000 cps |

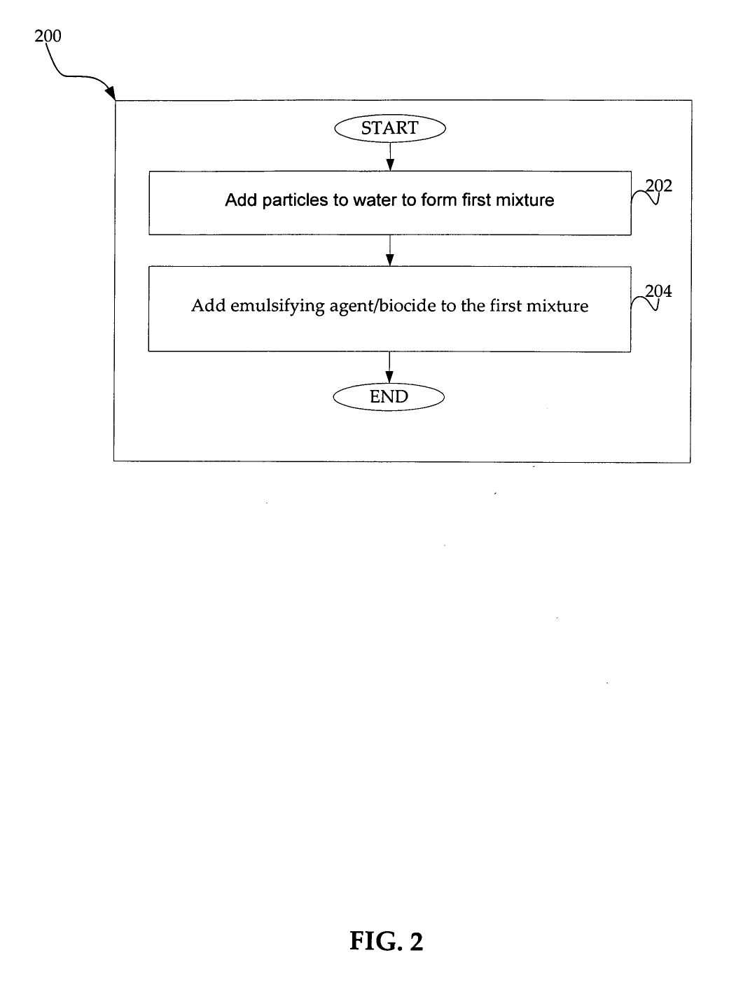

FIG. 2 is a flow chart of a second exemplary method 200 for making an exemplary sunscreen formulation for use in the production of organic crops.

At step 202, particles of low solubility, in the form of a micronized powder having a particle size distribution with a mean from about 0.01 to about 100 microns (and preferably from about 0.1 to about five microns), are added to about fifteen to about fifty-nine weight percent of water. The particulate material should be completely wetted and dispersed in the mixture. Optionally, the mean particle size may be reduced to less than one micron before or after performing this step (e.g., by wet milling).

At step 204, from about 0.01 to about five weight percent of a combined emulsifying agent and biocide (or preservative) is added to the mixture of step 202 to form a uniform aqueous suspension. Please note: standard methods of mixing, suspension, and dispersion may be used at each of the above steps.

The method exemplified in FIG. 2 may also include additional steps for sterilization, such as sterilizing the particles by exposing them to chemicals such as ethyl alcohol, hydrogen peroxide, ethanol and/or gaseous ozone prior to performing the method of step 202. Additionally, the particles may be heated to a temperature of at least 30 degrees Centigrade, and preferably to a temperature of at least 90 degrees Centigrade before performing the method of step 202. The various mixtures described herein may be exposed to ozone, ultraviolet light, pasteurization processes, and/or other sterilization techniques. Packaging materials may also be sterilized.

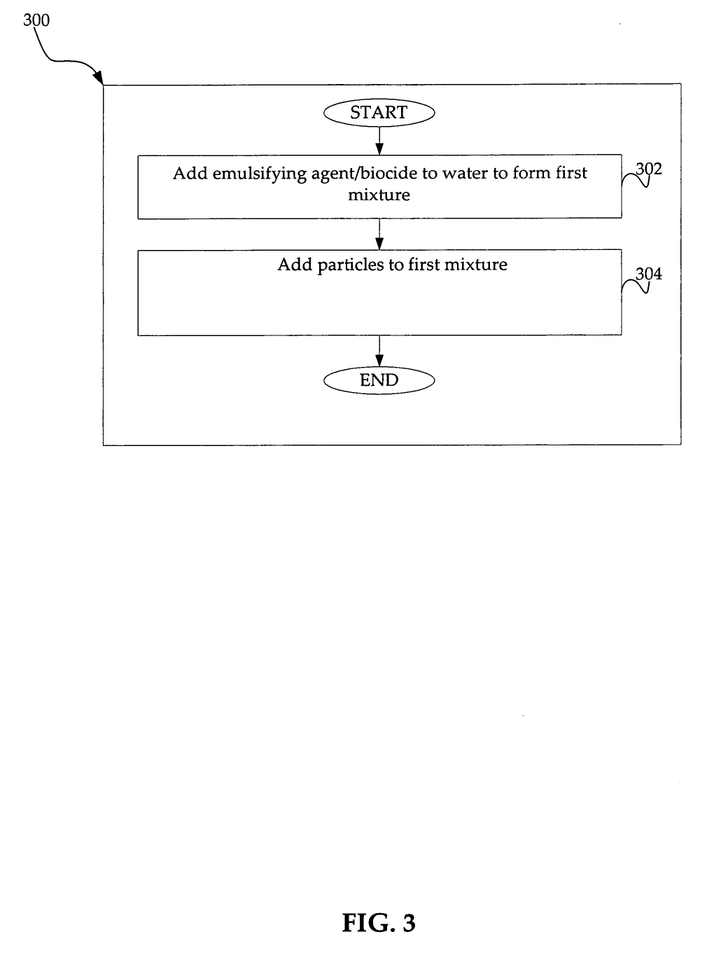

FIG. 3 is a flow chart of a third exemplary method 300 for making an exemplary sunscreen formulation for use in the production of organic crops.

At step 302, from about 0.01 to about five weight percent of a combined emulsifying agent and biocide (or preservative) is added to about fifteen to about fifty-nine weight percent of water to form a first mixture.

At step 304, particles of low solubility, in the form of a micronized powder having a particle size distribution with a mean from about 0.01 to about 100 microns (and preferably from about 0.1 to about five microns), are added to the first mixture. The particulate material should be completely wetted and dispersed in the mixture. Optionally, the mean particle size may be reduced to less than one micron before or after performing this step (e.g., by wet milling). The mixture of step 304 should form a uniform aqueous suspension. Please note: standard methods of mixing, suspension, and dispersion may be used at each of the above steps.

The method exemplified in FIG. 3 may also include additional steps for sterilization, such as sterilizing the particles by exposing them to chemicals such as ethyl alcohol, hydrogen peroxide, ethanol and/or gaseous ozone prior to performing the method of step 304. Additionally, the particles may be heated to a temperature of at least 30 degrees Centigrade, and preferably to a temperature of at least 90 degrees Centigrade before performing the method of step 304. The various mixtures described herein may be exposed to ozone, ultraviolet light, pasteurization processes, and/or other sterilization techniques. Packaging materials may also be sterilized.

Table 2 shows another exemplary agricultural sunscreen formulation. All percentages are shown on a weight/weight (grams/grams) basis. Some or all of the ingredients may comply with certification requirements for organic crop production. Such a sunscreen formulation may incorporate a minimum number of ingredients to result in an aqueous suspension concentrate. In the exemplary sunscreen formulation shown in Table 2, sodium bicarbonate functions as both an emulsifying agent and a biocide (or preservative). Other ingredients with similar properties may also be used.

TABLE 2

| Ingredient | Percent w/w |
|---|---|
| Calcium Carbonate | 40-80 |
| Sodium Bicarbonate | 1-5 |
| Water Q.S. | 100 |

The various exemplary agricultural sunscreen formulations described herein may be shipped to a final destination in the form of an aqueous suspension concentrate and applied to plant tissues in a variety of ways. For example, an exemplary aqueous suspension concentrate may be diluted in water using a ratio of one part aqueous suspension concentrate to at least one part of water (or a ratio of three parts aqueous suspension concentrate to 100 parts water) and applied to plant tissues by spraying.

Some or all of the various exemplary agricultural sunscreen formulations described herein may be applied to a variety of plant tissues. For example, mature fruit trees (e.g., apple trees) may be sprayed with a sunscreen formulation at least once while some or all of the fruit is developing. Immature trees or plants growing in a nursery may be sprayed with a sunscreen formulation prior to and/or following transplantation. Row crops such as grapes, tomatoes, peppers and onions may be sprayed at least once prior to harvest. Additionally, a sunscreen formulation may be applied to the ground surrounding the plant tissues to be sprayed. Reference is made to U.S. Provisional Patent Application Ser. No. 61/003,782 filed on Nov. 19, 2007 titled “Using Particle Films to Reduce Stress in Plant Tissue,” and U.S. patent application Ser. No. ______ filed on Nov. ______, 2008 titled “Systems and Methods for Applying Particle Films to Control Stress on Plant Tissues,” both of which are incorporated herein by reference for their teachings regarding the application of agricultural sunscreen formulations.

Table 3 shows some exemplary emulsion and/or preservation formulations for use in an agricultural sunscreen formulation suitable for the production of an organic crop. All percentages are shown on a weight/weight (grams/grams) basis. Some or all of the ingredients may comply with certification requirements for organic food production and/or organic crop production. It is assumed the exemplary emulsion and/or preservation formulations shown in Table 3 may contain from about 40 to about 80 percent weight/weight of calcium carbonate, and preferably from about 50 to about 70 percent weight/weight of calcium carbonate. It is also assumed the exemplary emulsion and/or preservation formulations shown in Table 3 may contain from about 10 to about 60 percent weight/weight of water, and preferably from about 20 percent to about 40 percent weight/weight of water.

Some of the challenges in creating a sunscreen formulation suitable for use in the production of organic crops include innovating a formulation that allows it to be manufactured, stored and shipped to customers with little or no degradation, maintained as a stable and non-perishable emulsion, applied with currently available spray equipment, and capable of achieving effective coverage of plant tissues. The exemplary emulsion and/or preservation formulations shown in Table 3 facilitate that some or all of the herein described challenges are satisfied or exceeded. For instance, a sunscreen formulation suitable for use in the production of an organic crop should maintain a consistent distribution of particles throughout its aqueous suspension over an extended period of time. It is undesirable for the particles to settle to a portion of the aqueous suspension. The aqueous suspension needs to be such that it does not foster the growth of contaminants, such as bacteria or viruses over an extended period of time (e.g., 3 months to 5 years). Accordingly, a suitable preservative may be included as part of some of the formulations shown in Table 3. If the aqueous suspension includes an organic ingredient that may foster the growth of contaminants, a biocide may be included as part of some of the formulations shown in Table 3 to avoid the growth of contaminants over an extended period of time.

TABLE 3

| Ingredient | Percent w/w |
|---|---|
| Formulation 1: | |
| Smectite Clay | 1.0-3.0 |
| Formulation 2: | |
| Lecithin (organic soy-based) | 0.5-5.0 |
| Sodium Bicarbonate | 0.5-5.0 |
| Formulation 3: | |
| Lignin Sulfonate (plant pulp) | 1.0-3.0 |
| Formulation 4: | |
| Smectite Clay | 1.0-3.0 |
| Sodium Bicarbonate | 0.5-5.0 |
| Formulation 5: | |
| Xanthan Gum | 0.1-0.5 |
| Glycerin | 0-8.0 |
| Sodium Bicarbonate | 0.5-5.0 |
| Formulation 6: | |
| Lecithin (organic soy-based) | 0.5-5.0 |
| Bacillus subtilis | 0.0-5.0 |
| Formulation 7: | |
| Xanthan Gum | 0.1-0.5 |
| Glycerin | 0-8.0 |
| Bacillus subtilis | 0.0-5.0 |

Some or all of the exemplary emulsion and/or preservation formulations shown in Table 3 may be used in agricultural sunscreen formulations suitable for the production of an organic crop wherein other ingredients are used in place of or in addition to calcium carbonate particles. Some or all of the ingredients may comply with certification requirements for organic food production and/or organic crop production. It is assumed the exemplary emulsion and/or preservation formulations shown in Table 3 may contain from about 40 to about 80 percent weight/weight of the ingredients used in place of or in addition to calcium carbonate particles, and preferably from about 50 to about 70 percent weight/weight of such ingredients. These ingredients may include finely divided calcined kaolins, hydrophobic calcined kaolins, hydrous kaolins, hydrophobic hydrous kaolins, and/or mixtures thereof. It is also assumed the exemplary emulsion and/or preservation formulations shown in Table 3 may contain from about 10 to about 60 percent weight/weight of water, and preferably from about 20 percent to about 40 percent weight/weight of water.

While various embodiments have been described above, it should be understood that they have been presented by way of example only, and not limitation. Thus, the breadth and scope of a preferred embodiment should not be limited by any of the herein-described exemplary embodiments.

What is claimed is:

1. An agricultural sunscreen formulation for use in production of an organic crop, the agricultural sunscreen formulation comprising on a weight to weight basis:
about 40% to 80% calcium carbonate;
about 1% to 5% sodium bicarbonate; and
about 15% to 59% water.

2. The agricultural sunscreen formulation of claim 1, wherein the agricultural sunscreen formulation has a viscosity from about 1,000 to about 45,000 centipoise.

3. An agricultural sunscreen formulation for use in production of an organic crop, the agricultural sunscreen formulation comprising on a weight to weight basis:
about 40% to 80% calcium carbonate;
about 0% to 8% glycerin;
about 0.1% to 0.5% potassium bicarbonate; and
about 11.5% to 59.9% water.

4. The agricultural sunscreen formulation of claim 3, wherein the agricultural sunscreen formulation has a viscosity from about 10,000 to about 35,000 centipoise.

5. A method for making an agricultural sunscreen formulation for use in production of an organic crop, the method comprising:
combining a first amount of glycerin and an amount of water to form a first mixture;
combining an amount of particles with the first mixture;
combining an amount of a thickening agent and a second amount of glycerin to form a second mixture; and
combining the first and second mixtures.

6. The method of claim 5, wherein the first amount of glycerin is from about zero to about three weight percent.

7. The method of claim 5, the method further comprising:
combining an amount of a preservative or a biocide with the first mixture.

8. The method of claim 7, wherein the amount of the preservative or the biocide is from about 0.01 to about five weight percent.

9. The method of claim 5, wherein the particles have a low solubility.

10. The method of claim 5, wherein the particles are in a form of a micronized powder having a particle size distribution with a mean from about 0.01 to about 100 microns.

11. The method of claim 5, the method further comprising:
sterilizing the particles before combining with the first mixture.

12. The method of claim 5, the method further comprising:
exposing the particles to ozone before combining with the first mixture.

13. The method of claim 5, the method further comprising:
exposing the particles to ozone before combining with the first mixture.

14. The method of claim 5, wherein the amount of the thickening agent is from about zero to about five weight percent.

15. The method of claim 5, the method further comprising:
combining an amount of water with the second mixture.

16. The method of claim 15, wherein the amount of water is from about zero to about eight weight percent.

17. The method of claim 5, the method further comprising:
combing the first mixture with an amount of a wetting agent.

18. The method of claim 5, the method further comprising:
combining the first mixture with an amount of a dispersion agent.

19. The method of claim 5, the method further comprising:
combining the first mixture with an amount of an emulsifier.

20. A method for making an agricultural sunscreen formulation for use in production of an organic crop, the method comprising:
combining about 40% to 80% calcium carbonate with about 1% to 5% sodium bicarbonate and about 15% to 59% water to form an aqueous suspension.

21. A substrate comprising plant tissues selected from a group consisting of fruits, vegetables, trees, flowers, grasses, roots and landscape and ornamental plants wherein the plant tissues are at least partially coated with the agricultural sunscreen formulation of claim 1.

22. A substrate comprising plant tissues selected from a group consisting of fruits, vegetables, trees, flowers, grasses, roots and landscape and ornamental plants wherein the plant tissues at least partially coated with the agricultural sunscreen formulation of claim 3.

23. A formulation for emulsion and preservation of an agricultural sunscreen formulation for use in production of an organic crop, the formulation for emulsion and preservation comprising on a weight to weight basis:
about 0.5% to 5.0% lecithin; and
about 1% to 4% sodium bicarbonate.

24. The formulation for emulsion and preservation of claim 23, the formulation further comprising:
about 40% to 80% kaolin clay.

25. A substrate comprising plant tissues selected from a group consisting of fruits, vegetables, trees, flowers, grasses, roots and landscape and ornamental plants wherein the plant tissues at least partially coated with the agricultural sunscreen formulation of claim 24.
